SEQUENCE LISTING (6 sequences)

SEQ ID NO: 1 - protein, 20 aa, Artificial Sequence
  Peptide Ligand
LLYNKTFPHGRWSPSYPGSC

SEQ ID NO: 2 - protein, 20 aa, Artificial Sequence
  Peptide Ligand
DWARSNTSRSMDFNLGWGSC

SEQ ID NO: 3 - protein, 20 aa, Artificial Sequence
  Peptide Ligand
SFTFNWLKTDSKSGMHGGSC

SEQ ID NO: 4 - protein, 20 aa, Artificial Sequence
  Peptide Ligand
LFFNIWPRRDPYWPAAWGSC

SEQ ID NO: 5 - protein, 20 aa, Artificial Sequence
  Peptide Ligand
YIGTQIRVHWPANPHPVGSC

SEQ ID NO: 6 - protein, 20 aa, Artificial Sequence
  Peptide Ligand
RWHRVDLRSHTELPRYIGSC

(19) United States
(12) Patent Application Publication
(10) Pub. No.: US 2013/0224730 A1
(43) Pub. Date: Aug. 29, 2013

(54) PEPTIDE LIGANDS

(71) Applicants: Stephen A. Johnston, Tempe, AZ (US); Christopher Diehnelt, Chandler, AZ (US); Paul Belcher, Boston, MA (US); Charles J. Arntzen, Gold Canyon, AZ (US); Robert Sutherland, Gilbert, AZ (US)

(72) Inventors: Stephen A. Johnston, Tempe, AZ (US); Christopher Diehnelt, Chandler, AZ (US); Paul Belcher, Boston, MA (US); Charles J. Arntzen, Gold Canyon, AZ (US); Robert Sutherland, Gilbert, AZ (US)

(73) Assignee: ARIZONA BOARD OF REGENTS ACTING FOR AND ON BEHALF OF ARIZONA STATE UNIVERSITY, SCOTTSDALE, AZ (US)

(21) Appl. No.: 13/510,161

(22) PCT Filed: Nov. 16, 2010
(86) PCT No.: PCT/US10/56809

§ 371 Date: Jan. 4, 2013

Related U.S. Application Data
(60) Provisional application No. 61/262,385, filed on Nov. 18, 2009.

(51) Int. Cl. C12N 7/02; C07K 1/22; C12Q 1/70

(52) U.S. Cl. CPC C12N 7/02; C12Q 1/701; C07K 1/22
USPC 435/5; 435/239; 506/9; 530/326; 530/344

(57) ABSTRACT

A method of solid phase selection of peptide ligands for target proteins is presented. 15-20mers or greater are addressed in a microarray, and the target protein and optional competitor bound thereto and binding compared. A specific signal for the target protein indicates that a peptide has strong affinity for the target. Ligands can be coupled to solid supports and used for affinity purification of the target proteins as well as detection and modulation of target proteins. Specific peptide ligands for immuno-purifying norovirus.

### CROSS-REFERENCE TO RELATED APPLICATIONS

Not applicable.

### FEDERALLY SPONSORED RESEARCH STATEMENT

Not applicable.

### REFERENCE TO MICROFICHE APPENDIX

Not applicable.

### FIELD OF THE INVENTION

This invention relates to a novel method of rapidly selecting peptide ligands for affinity purification, detection assays, target modulation and other uses. Particular ligands with high affinity to Norovirus and their use to affinity purify or detect norovirus are also provided.

### BACKGROUND OF THE INVENTION

The norovirus was originally named the “Norwalk virus” after an outbreak of acute gastroenteritis occurred among school children in Norwalk, Ohio in 1968. “Norovirus” abbreviated “NV,” was recently approved as the official genus name for the group of viruses provisionally described as “Norwalk-like viruses.” This group of viruses has also been referred to as caliciviruses (because of their virus family name Caliciviridae) and as small round structured viruses (because of their morphologic features). Norovirus causes almost 90% of epidemic, non-bacterial outbreaks of gastroenteritis around the world, but the general public often refers to this illness as “stomach flu,” even though the illness is not influenza related.

The norovirus are non-enveloped, icosahedral viruses with a positive-sense, single-stranded RNA genome of ˜7.5 kb that contains three open reading frames (ORFs). ORF1 encodes a polyprotein that is further cleaved into at least six nonstructural (NS) proteins by the viral 3C-like protease. ORF2 encodes the major capsid protein (norovirus capsid protein; NVCP), or viral protein 1 (VP1), which consists of a shell domain (S domain) and a protruding domain (P domain). The S domain is more highly conserved than the P domain. The P domain is subdivided into the P1 and P2 subdomains, with the hypervariable P2 subdomain containing both immune and cellular recognition sites. ORF3 encodes the small basic protein, or VP2, which is found in the virion and has a role in virion stability.

The lack of a permissive cell culture system and animal model has prevented the serotyping of human noroviruses. Consequently, genetic analysis has been used to classify these viruses. Five genetic groups, or genogroups, of noroviruses have been identified using phylogenetic analysis of VP1 sequences. Genogroups I, II, and IV contain human noroviruses, while genogroups III and V contain bovine noroviruses and murine noroviruses (MNVs), respectively. Genogroup II also contains porcine noroviruses. Genogroups I, II, and IV can be further subdivided into 29 distinct phylogenetic clusters, or genotypes.

Human noroviruses are genetically diverse; over 100 strains have been sequenced to date. Full-length human norovirus genomes diverge by as much as 45% at the nucleotide level, while VP1 sequences diverge by as much as 57%. In 1995 and 2002, genogroup II/4 noroviruses emerged and subsequently spread globally to become the dominant norovirus strain.

The virus is extremely infectious, with as few as 10 virions able to cause illness. The symptoms include acute diarrhea and vomiting, abdominal cramps, headache, nausea, fatigue, and low-grade fever. In most cases, the illness is resolved within 24-48 hours without long-term medical consequence, but occasionally mortalities do occur in the young, elderly, and immuno-compromised, as a result of complications brought on by dehydration.

In spite of the very high prevalence of norovirus infections, there is still no vaccine available to prevent the disease, and progress is hindered by the difficulty of growing the virus in culture and the absence of a suitable animal model for preclinical testing of vaccine candidates. Norovirus will not reproduce in a simple growth medium and has resisted growth in cell culture. Thus, most virus was cultured in human volunteers and then isolated from the stools for many years. However, the capsid protein of norovirus has been sucessfully expressed in plant and insect cells, and these capsid proteins present another option for vaccine development. Viruses like particles or “VLPs” can be assembled from the capsid structural subunits to antigenically resemble the native virus. However, they lack viral nucleic acid, rendering them non-infectious and an excellent alternative for vaccine development.

Expression of NVCP in insect cells yields a protein with an apparent molecular weight of 58,000 (58 kDa) that self-assembled into nVLPs lacking viral RNA (Jiang, 1992). Electron cryomicroscopy of the nVLPs showed that the 38 nm empty capsid was composed of 90 dimers of NVCP that form arch-like capsomeres (Prasad, 1994). The particles were morphologically and antigenically similar to authentic virus particles, but non-infectious, stable on storage at 4° C., stable after lyophilization, and resistant to pH 3.0 treatment (Jiang, 1992; Green, 1993). These qualities make these nVLPs attractive for use as a potential vaccine against Norwalk virus. Indeed, studies have shown that oral immunization of mice with as little as 50 μg nVLPs per dose resulted in the production of serum and mucosal antibodies against NVCP (Ball, 1995). This result is striking in view of the fact that the particle is a non-replicating vaccine and no cholera toxin (CT) adjuvant is needed to achieve immunization.

With these encouraging results, many laboratories are striving to develop a cost effective and efficacious norovirus vaccine. For example, U.S. Pat. No. 6,942,865 describes norovirus VLPs production from insect cell culture, while US2005255480 and US2008254443 describe production in a permissive cell cultures, and WO2005032245 describes production in plant cells. WO2006138514 describes vaccine production and efficacy, as does WO2009039229 and US20080299152.

While norovirus like particles (nVLPs) have been produced in a variety of prokaryotic and eukaryotic heterologous expression systems, purification of the recombinant proteins remains an obstacle for cost effective vaccine development. Current nVLP isolation techniques usually use a series of non-specific column separation techniques and/or sucrose gradient purification. As a result, a robust but cost effective process for purifying either the noroviruses or VLP vaccine candidates to the high level of purity required for clinical trials does not exist, and the existing purification methods can be greatly improved in terms of yield, purity, and cost effectiveness.

A method of selecting for peptide or peptide-like ligands having high affinity and selectivity for the norovirus particle would therefore be of great benefit in improving affinity purification of the virus, and allow easier, less expensive production of virus for further research, vaccine development and development of robust point of care detection assays.

A method of selecting high affinity peptide ligands would have broad applicability beyond just norovirus VLP production, however. For other VLP vaccines, such as Gardisil (to prevent human papiloma virus infection) or virus-like-particle H1N1 vaccines, purification costs are a significant factor and the use of peptide ligands for affinity purification could provide significant cost savings. Peptide ligands could also be used to purify recombinant or natural proteins from any source, have uses in detection reactions, and some may have uses in modulating target activity. Peptide ligands for specific complexes would also be useful in similar ways.

Peptide ligand selection techniques of several kinds are already available in the art. For example, phage display and the two hybrid system have been used to select peptide ligands for various uses. These techniques provide some advantage, because sequential application of the bind and wash steps followed by regrowth of the bound phage or yeast greatly enriches the library for active ligands. However, sequential screening adds to the time required for the technique, and all such techniques are limited in that any ligand that inhibits cell or vector growth will be selected against and disappear from the library. Additionally, because cell/phage viability needs to be maintained, the wash techniques are typically very mild, leading to very high false positive rates and resulting in the need to screen enormous numbers of ligands, typically on the order of 10⁶-10⁹. Further, the need for specific and applicable reporters hampers universal application of these methods.

U.S. Pat. No. 7,217,507 describes a method of peptide trapping, whereby ligands are immobilized in a gel, bind to target and then capillary transferred to a membrane and target detected. Placement of the detected target on the membrane indicates where in the original gel the binding ligand could be found. The ligand can be collected therefrom and tested a second time for target binding. In this instance, peptide sequencing reactions were then used to identify the random hexamer ligands. The method has advantages because neither the target nor the ligand are pre-labeled, and thus their interaction proceeds without interference from any labelling group. However, the method still requires a specific detection method for each target protein, and the gel transfer makes the system cumbersome and slow. Further, although the specification states that the method can be used with up to 15 mers, the invention is only exemplified with hexamers. Therefore, the ligands are quite short and likely to be of limited functionality. Finally, the method is suspectible to a high rate of false positives, although competitive binding to target molecules is also taught and may help to somewhat reduce the false positives.

U.S. Pat. No. 5,834,318 describes a column based peptide ligand trapping method wherein a peptide library is bound to chromatographic supports and then incubated with a first detecting agent, resulting in e.g., a color change where ever the detecting agent binds the peptides or the support. Without washing the column, the target protein is then added to the supports under desired binding conditions. A detection reagent specific for the target protein is again added, this time resulting in a different color change. Beads having the second color are then isolated from the rest, and the peptide eluted and sequenced. The method is very cumbersome however, because of the need to isolate colored beads and the need for peptide sequencing. Further, the color detection methods exemplified have the strong possibility of interfering with target-ligand binding, and as for most techniques target specific detection methods are needed for the technique.

What is needed in the art is a peptide ligand selection or trapping technique that has universal applicability, is not hindered by bulky labeling groups, is quick and easy, and yet reduces the high rate of false positives that can make screening large libraries so tedious. It should also be inexpensive, not consume large amounts of target sample and preferably be in vitro to afford more flexibility in selection conditions.

### SUMMARY OF THE INVENTION

The following abbreviations are used herein:

| | |
|---|---|
| AKT1 | V-AKT MURINE THYMOMA VIRAL ONCOGENE HOMOLOG 1 aka |
| | PROTEIN KINASE B-ALPHA, PKB-ALPHA, RAC SERINE/THREONINE |
| | PROTEIN KINASE |
| BSA | bovine serum albumin |
| CCD | Charge-Couple Devices |
| CMOS | complementary metal-oxide semiconductor |
| DEAE | diethylaminoethyl cellulose |
| FET | FETUIN; aka ALPHA-2-HS-GLYCOPROTEIN; AHSG |
| | A2HS; AHS; HSGA |
| FMOC | fluorenylmethyloxycarbonyl chloride |
| HBS-N | Hepes buffered saline, 10 mM Hepes (pH 7.4) 150 mM sodium |
| | chloride |
| HPLC | high performance liquid chromatography |
| HRP | horse radish peroxidase |
| MALDI | matrix-assisted laser desorption/ionization |
| MES | 2-(N-morpholino)ethanesulfonic acid |
| nVLP | norovirus-like particles |
| PBS | phosphate buffered saline (3.2 mM Na2HPO4, 0.5 mM KH2PO4, 1.3 mM KCl, |
| | 135 mM NaCl, 0.05% pH 7.4.) |
| PBST | phosphate buffered saline TWEEN ®-20 (3.2 mM Na2HPO4, 0.5 mM KH2PO4, |
| | 1.3 mM KCl, 135 mM NaCl, 0.05% TWEEN ® 20, pH 7.4.) |
| PMSF | phenylmethanesulphonylfluoride |
| PMT | photomultiplier tube |
| RT | residence time |
| SPR | surface plasmon resonance |
| TBST | Tris buffered saline TWEEN-20 ®, 10 mM Tris-HCl, 150 mM NaCl, 0.1% |
| | TWEEN ®-20, pH 7.5 |
| TFA | trifluoroacetic acid |
| TFF | tangential flow filtration |
| TIPS | tri-isopropyl silane |
| TNFA | TUMOR NECROSIS FACTOR, ALPHA, aka CACHECTIN |

The use of the word “a” or “an” when used in conjunction with the term “comprising” in the claims or the specification means one or more than one, unless the context dictates otherwise. The term “about” means the stated value plus or minus the margin of error of measurement or plus or minus 10% if no method of measurement is indicated. The use of the term “or” in the claims is used to mean “and/or” unless explicitly indicated to refer to alternatives only or if the alternatives are mutually exclusive. The terms “comprise”, “have”, “include” and “contain” (and their variants) are open-ended linking verbs and allow the addition of other elements when used in a claim.

The term “peptide ligand” as used herein is intended to encompass peptide ligands having normal peptide backbones and amino acid constituents, as well as modified backbones and non-natural amino acids. Thus, the term includes peptidomimetics that are altered to improve stability and resistance to protease. Peptide ligands may also be coupled to labeling or detection reagents, such as ALEXA FLUOR® or biotin, and the like, and to solid supports. Because the term peptide ligand includes peptidomimetics, reference to a SEQ ID NO herein, means that the peptide ligand contains the same side chains in the same order as found in the referenced SEQ ID NO., even though the backbone may be altered.

The term “norovirus” as used herein includes norovirus and norovirus like particles, including uni- and multivalent virus like particles.

The term “screening” as used herein means that the target protein (or competitor) is applied to a microarray under suitable binding conditions, and non-specific binding is washed away to leave specific binding of said target protein to said microarray.

The term “microarray” as used herein means a ordered array of peptide ligand candidates each placed at known positions on a solid support. The solid support can be any solid support used in microarray production, but typically is a glass or plastic slide, a well plate, or a silicon chip.

The invention generally relates to a method of identifying peptide or peptide-like ligands from a library of ligands with greatly reduced false positive rate and without interference from bulky labeling groups.

The core technology involves screening a protein target against a library of about 10,000 peptides that are at least 15 amino acids long, preferably 20 amino acids long, and of random sequence. Peptides of this length have not been used in the art because it is generally believed that too many peptides would need to be screened to screen all possible 20 mer peptides. The peptides are usually spotted in an addressable fashion on a slide or chip. We have surprisingly discovered that fewer peptides need to be screened because 10,000 20 mers include all possible dimers and trimers several times over, and even include some 60% of all possible tetramers. Thus, a small number of peptides has a surprisingly large collection of smaller peptides contained therein.

In addition, the longer sequences provide opportunities for more contact points with the target and thus increased binding strength. The increased affinity provides several advantages. First, smaller amounts of target protein can be used, on the level of microgram quantities, instead of milligram quantities in most prior art methods. Second, the increased affinity results in fewer false positives and therefore fewer peptides need be screened. Third, the increased affinity (and lack of cellular or viral components in the methodology) means that more stringent wash conditions can be imposed, again reducing false positives and reducing the number of peptides that need to be screened.

One important aspect of the methodology is that the random sequence peptides are generally unstructured and make linear contacts with the target protein. This feature means the peptides are not subject to denaturation, so the affinity ligand is much more stable than a structure based reagent such as antibodies, aptamers, affibodies, etc. This feature also allows the use of more stringent wash conditions, again reducing the number of false positives.

In preferred embodiments, the library contains 1,000-10,000 peptides, but larger libraries of 20,000, 50,000, 100,000 or more peptides can be screened if desired. Further, we have exemplified 20 mers herein, but slightly shorter or longer sequences can also be used. Thus, 15, 16, 17, 18, 19, 20, 21, 22, 23, 24, and 25 mers and up can be used in the invention, although sequences of at least 15 mers or 20 mers are preferred.

In a preferred embodiment, the ligands are addressed at known locations on a solid support for screening, such as a microscope slide. Because the ligands are screened while immobilized, it is guaranteed that the chosen ligand will be coupled to the purification matrix through a reactive moiety that plays no role in the binding domain to the target. This helps to eliminate those false positives in the prior art that may bind in solution, but lose their affinity for the target when coupled to an affinity support. Thus, with fewer false positives, smaller libraries need be screened to identify ligands with high affinity.

Thus, the inventive method provides several different mechanisms for reducing the high false positive rate, and makes the method quicker and easier than the prior art methods. Indeed, we have discovered and validated several norovirus peptide ligands from a dual source VLP screening of a 10,000 peptide library in a period of two months. This counterintuitive result is an enormous improvement over prior art methodologies.

In addition to using larger peptide ligands, some embodiments of the method also employ a competitor to further reduce the rate of false positives. We used E. coli lysates as a competitor herein because E. coli has about 5000 proteins and the proteins are roughly equally presented. Therefore, the bacterial cell lysate provides a rigorous collection of peptide competitors. Of course, the lysate of any bacteria will equally suffice in the method, but E. coli is a preferred competitor, because most recombinant proteins are made in E. coli. Other possible competitors include BSA or other proteins, and plant, insect or mammalian cell line lysates, which may be preferred where the target protein (or virus) is made in plant cells, insect cells or in cell culture.

In some embodiments, the target may be mixed with the competitor for the screening. In other cases, the target protein is present in the cell lysate from which it needs to be purified and this is directly applied to the slide, and target binding is detected with e.g., an antibody. In still other embodiments, duel screening with a target-cell lysate mixture from two different sources is used, and target binding is detected. The dual source method allows detection with polyclonal antibodies, since non-specific binding is expected to differ in the two sources, and thus, signal that is detected in both sources is likely to be a true signal. In other embodiments, a highly specific monoclonal antibody is used for labeling, and only one source is used for the screening.

In one embodiment of the invention, about 10,000 20 mers are addressed on a microarray, labeled competitor added to the array under binding conditions, the array washed and signals detected. A second array (or the same array) is bound with labeled target, washed and again signals detected. Peptide ligands will bind strongly to the target, but only weakly if at all to the competitor, and thus peptide ligands are quickly identified, with very few false positives.

In certain embodiments, the competitor and target need not be labeled for the screening. Instead, it is possible to label the target after binding to the microarray, e.g., with an antibody. Further, the competitor need not be separate from the target, but can be admixed therewith, as might be common where a cell lysate is applied to the microarray for screening. In such case, the competitor need not be labeled, but it can be labeled if one desires to know the degree of comparative binding. Such label can be a polyclonal antibody, directed to the lysate in without said target present, or can be a nonspecific protein labeling method, such as Coomassie blue, silver stain, in addition to the various labels described below.

In preferred embodiments the competitor and target protein are prelabeled with different radiolabels or fluorescent dye. Radiolabels have previously been avoided in an effort to avoid consuming and disposing of large quantities of dangerous radioactive chemicals. However, with the advent of microarrays and CCD detector technology, very small amounts of radiolabels can be used and their popularity is again returning. These labels may be preferred because their small size minimizes interference with ligand affinity binding to the target. Suitable isotopic labels include C¹³, C¹⁴, H², H³, S³⁵, O¹⁷, O¹⁸ and the like.

However, for many scientists fluorescent dyes are preferred because many labs are already equipped with fluorescence detectors. Thus, dyes such as acridine dyes, cyanine dyes, fluorone dyes, oxazin dyes, phenanthridine dyes and rhodamine dyes, as well as biological fluors such as luciferin and green fluorescent protein can all be used in the invention. For example, the ALEXA FLUOR™ dyes come in a wide range of colors, and are preferred by many.

Additionally, labels can be antibody based, meaning that the target protein need not be purified in the screening method. In fact, our experiments with the norovirus were performed with virus that was only 60% purified, and thus contained substantial impurities.

Other common detection methods include colorimetric techniques based on silver-precipitation, chemiluminescent and label free techniques, such as Surface Plasmon Resonance.

The microarray can be produced by any of the numerous methods known in the art, including external synthesis and attachment to the array or in situ chemical or biological synthesis, such as in vitro translation in situ. One preferred means of making an array is external synthesis and ink jet printing onto an array surface. In the examples described, the peptides were printed with a quill-based contact spotter.

In other embodiments of the invention, the peptide ligands are used for affinity purification of target proteins by coupling the peptide ligand to a solid support, applying a sample to the solid support under binding conditions, washing the solid support, and eluting purified target.

In other embodiments, the peptide ligands are used for a target detection assay, wherein a sample is contacted with a peptide ligand under binding conditions, said sample is washed to leave only specific binding of said peptide ligand to said target, wherein detecting specific binding of said peptide ligand to said sample indicates the presence of norovirus. The detection can be direct, by prelabeling the ligand, or can be indirect, e.g., by subsequent antibody detection, and the like. In preferred embodiments, the peptide ligand is coupled to a solid support for ease in washing. In other embodiments, the peptide ligands can be spotted onto a dip stick for a lateral diffusion type of detection assay.

FIG. 1 shows a schematic of peptide selection on arrays. The target protein is labeled, for example, with a green fluorescent dye, while the competitor is labeled with a red fluorescent dye and the ratio of the intensity of the two colors indicates which peptide specifically binds the target protein. Technology already exists (e.g., CMOS cameras) to visualize fluorescence labels and quantify the signal. However, any small label that doesn't interfere with peptide target binding can be employed, e.g., radioisotopes and CCD detection.

This solid phase peptide ligand selection method has been successfully applied to identify peptide ligands for the following target proteins:

| Target Protein | Competitor Mixture |
|---|---|
| TF | E. coli lysate |
| AKT1 | E. coli lysate |
| TNFA | E. coli lysate |
| FET | E. coli lysate |
| Ubiquitin | E. coli Lysate |
| gp120 | E. coli lysate |
| NOROVIRUS | plant cell lysate |
| GAL 80 | BSA |

The peptides ligands discovered with our inventive approach generally had equilibrium dissociation constants (K_{D}) in the range of 10 to 200 μM measured by SPR. However, when such ligands were concentrated and multiplied by binding large numbers to a solid support, the effective binding affinity was greatly increased. Thus, ligands with micromolar affinity became nanomolar ligands when coupled to a solid support and could then be used for affinity purification and other techniques requiring high affinity peptide ligands.

### BRIEF DESCRIPTION OF THE DRAWINGS

FIG. 1 shows a schematic of one embodiment of the peptide ligand selection assay whereby 20 mers are addressed in a microarray, and prelabeled competitor and target are bound to the microarray and signals compared. A high target signal to competitor signal indicates a peptide ligand has been identified.

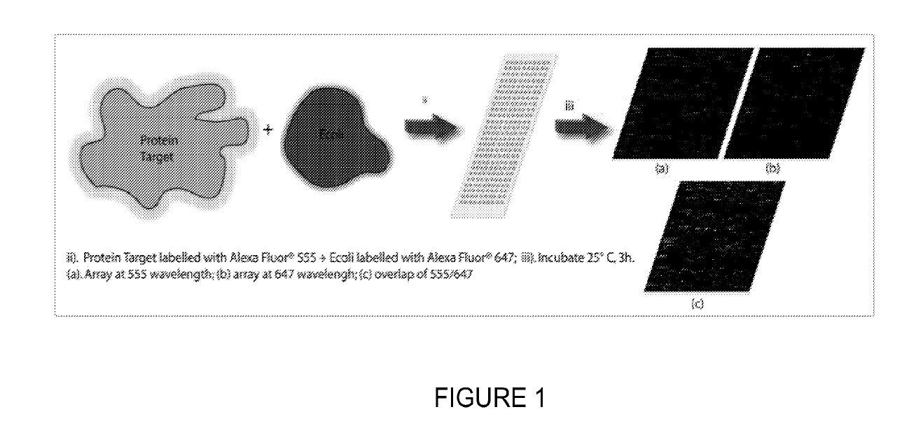

FIG. 2 shows a silver stained gel of AKT1 purification from 10 μg of E. coli lysate. AKT1 was loaded onto the peptide column and washed 5 times with 1× PBST and the pH 3.0 elution is shown in lane 5.

FIG. 3 shows a chematic of the ligand discovery process and use of concept to purify norovirus like particles.

FIG. 4 is a Venn diagram showing that a 10,000 peptide microarray was screened with a target/plant lysate mixture, and a target/insect lysate mixture, and target binding detected with a polyclonal antibody, which is then detected with a secondary antibody. Only those peptide ligands that give signal from insect and plant-expressed nVLP (99) in all three cases represent specific binding to target. Thus, many false positive signals are eliminated in this dual source screening embodiment of the invention.

FIG. 5 shows purification of crude nVLP on peptide affinity column. Protein samples were analyzed on NuPAGE™ 4-12% Bis-Tris gels in MES and visualized by Coomassie staining. Left hand gel—Lane 1: Crude nVLP as loaded onto column; Lane 2: load flow through; Lane 3: Combined wash fractions off column; Lane 4-9 Elution phase fractions. Right Hand gel condensed purified fractions. Lane 1: Crude nVLP as loaded onto column; Lane 2 Negative control with N. benthamiana leaf extract; Lane 3: Condensed fractions of purified nVLP.

FIG. 6 is a Western blot analysis of nVLP produced in N. benthamiana leaves, membrane probed with rabbit anti-nVLP detected with goat anti-rabbit HRP. Lane 1: Positive control GII.4 Narita reference; Lane 2: Negative control with N. benthamiana leaf extract; Lane 3: N. benthamiana expressing GII.4 Minerva VPI as an assembled nVLP via Agrobacterium-mediated transfection 200 μg total/lane. Lane 4: Purified nVLP.

### DESCRIPTION OF EMBODIMENTS OF THE INVENTION

The following examples are exemplary only and not intended to be limiting of the various embodiments of the invention.

EXAMPLE 1

### Peptide Ligand Library

Materials: Peptides were purchased from ALTA BIOSCIENCE™ (University of Birmingham, UK), polyacrylamide resin for coupling sulfhydryl-containing ligands (ULTRALINK™ iodoacetyl resin) was purchased from THERMO SCIENTIFIC™.

Peptide Ligand Library: Amino acids were selected at random in each of the first 17 positions with Gly-Ser-Cys-COOH as the C terminus linker. The synthesis scale was 2 μmole total at ˜70% purity with 2% of the peptides tested at random by mass spectrometry. Dry peptide was brought up in 100% dimethyl formamide until dissolved, then diluted 1:1 with purified water at pH 5.5 to a master concentration of ˜2 mg/ml. The original 96-deep-well plates were robotically transferred to 384-well spotting plates, and the peptides were adjusted to ˜1 mg/ml concentration in phosphate buffered saline at pH 7.2, before being further diluted 1:100 to give stock concentrations of around 50 μM. The peptides were robotically printed on to pre-activated aminosilane glass slides using a NANOPRINT LM60 CONTACT SPOTTER™ (ARRAYIT CORP.™, Sunnyvale Calif.).

### EXAMPLE 2

### Peptide Ligand Screening

An E. coli lysate was labeled by reacting the N-hydroxysuccinimidyltetrafluorophenyl ester of ALEXA FLOUR® 647 with the primary amines in the lysate using the manufacturer's recommended protocol. Each of the target proteins shown below was similarly labeled using ALEXA FLUOR® 555.

| Target Protein | Competitor Mixture |
|---|---|
| TF | E. coli lysate |
| AKT1 | E. coli lysate |
| TNFA | E. coli lysate |
| FET | E. coli lysate |
| Ubiquitin | E. coli lysate |
| gp120 | E. coli lysate |

The target and competitor were bound to the peptide ligands on the same slide with 100 nM of the target spiked into ˜100 fold excess competitor. Washes were performed with 1× TBST. Arrays were dried by centrifugation, and scanned on the PROSCAN ARRAY™ (PERKIN ELMER®) scanner at 555 and 647 nm and 70 PMT.

Using this technique, several ligands were identified with affinities for their target proteins in the micromolar range.

To further improve the affinities of the various peptide ligands, they were coupled to solid support materials shown below using the manufacturer's directions.

| Target Protein | Solid Support | Coupling Chemistry |
|---|---|---|
| FET | Tentagel (Thiol Beads) | Disulfide |
| FET | NHS-Sepharose | Malemide amine |
| FET | Tentagel | Direct Synthesis |
| TF | Tentagel | Disulfide |
| AKT1 | Ultralink | Iodoacetyl |
| AKT1 | Agarose | Malemide |
| TNFA | Ultralink | Iodoacetyl |

Therefore, the solid phase 20 mer ligand screening using cell lysates as a competitor efficiently generated ligands of micromolar affinity, which could easily be converted to nanomolar affinity by coupling to a support matrix. The method was both quick and easy, and the use of whole cell lysates as a competitor greatly reduced the number of false positives.

### EXAMPLE 3

### AKT1 Ligands

To prove that the peptide ligands isolated could be used in one step affinity purification, we selected a single peptide that bound AKT1 (K_{D}=12 μM) with little E. coli lysate binding and coupled it to ULTRALINK® beads using the recommended protocol.

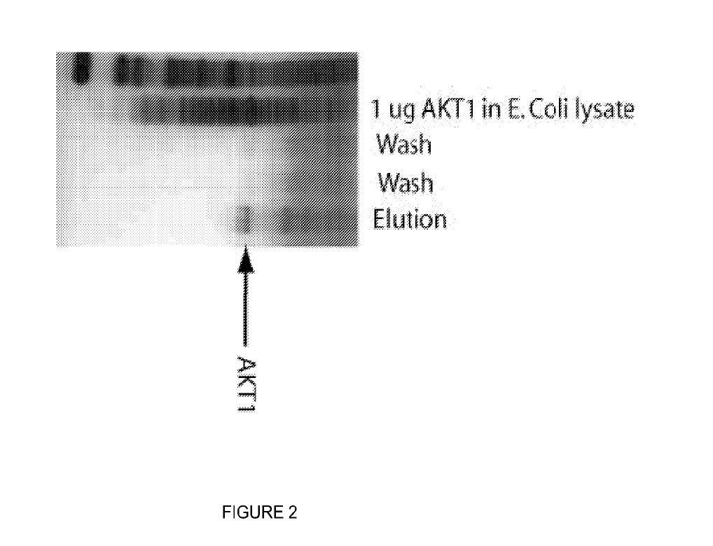

To test the performance of the ligand column, a 166 nM solution of recombinant AKT1 (1 μg total protein) was spiked into 10 fold excess E. coli lysate (by mass) and passed over the peptide column. The column was washed 5 times with 1× PBST and the bound AKT1 was eluted using 0.1 M glycine (pH 3.0). As can be seen in FIG. 2, AKT1 was selectively bound and eluted while no E. coli proteins were seen. Eluted proteins were quantified and the sample was 90% pure and overall, the column was estimated to have 90% recovery of loaded AKT1.

This level of purification was achieved with a single peptide ligand that had micromolar affinity before coupling to the solid support. However, we can further improve the purification yield in any of several ways. First, the affinity of the peptide for the target protein can be improved using the linear mutagenesis approach. Improvement in binding affinity should correspond to an improvement in binding specificity. We could also screen the peptide ligands under harsher conditions, so that peptides that stay bound under the more stringent wash conditions will be selected, and the use of more stringent wash conditions will generally improve purification. Another simple method of improving purification is to use more than one peptide ligand to purify the target protein. The selection procedure in each case identified multiple peptides that could bind the target protein and by using two different peptides with complimentary specificities, we should be able to easily increase the purity of the eluted protein. Experiments are planned to test this hypothesis, and we expect that purifications can easily reach 99% with two or more peptide ligands.

### EXAMPLE 3

### Norovirus

nVLP production: N. benthamiana (tobacco) plants were infiltrated with an Agrobacterium Ti plasmid encoding norovirus capsid protein according to known techniques. Biomass was harvested at 6 days post-infiltration and extracted using a GREEN STAR™ juicer and extraction buffer (25 mM sodium phosphate, 100 mM NaCl, 2 mM PMSF, 50 mM ascorbic acid, plus a PROTEASE INHIBITOR TABLET™ (SIGMA-ALDRICH®), pH 5.75).

The extract was incubated on ice for a minimum of 1 hour followed by centrifugation at 6,000×G for thirty minutes. The supernatant was filtered using a 0.8/0.2 micron capsule filter. The extract was incubated at 4° C. for 36 hours and the centrifugation process repeated as before. The extract was further incubated at 4° C. for 24 hours and the centrifugation process repeated again. The pH of the supernatant was adjusted after the third centrifugation to 7.30 using 0.25 M sodium phosphate and the centrifugation repeated again.

The final supernatant was concentrated using a 100 kDa cellulose acetate TFF membrane. The concentrate was loaded on a DEAE Sepharose column equilibrated in 25 mM sodium phosphate, 150 mM NaCl, pH 5.75, and the flowthrough collected into fractions at visible peak inflections (RT=23 min). The column was stripped with 25 mM sodium phosphate, 2 M NaCl, pH 5.75. ELISA testing indicated that noroviral capsid protein was present in Flow-Through Fraction 1 only. The Flow-Through Fraction 1 was clear, colorless, and particle-free in appearance.

Microarray Screening: Slides containing the library of peptide ligands were incubated for one hour in blocking buffer (1 mM Mercaptohexanol, 3% BSA, 0.5% Tween in 1× PBS) to passify the remaining free malimide groups. This and all subsequent incubations were preformed in a hybridization oven (AGILENT®) at 37° C. with rotation at 6 rpm. Slides were washed twice in TBST and once in diH2O. Insect and plant VLP were diluted to 10 μg/ml in incubation buffer (3% BSA, 0.5% Tween in 1× PBS) and 450 μL was added to each array. Thus, the dual competitors (plant and insect lysate) in this instance are admixed with target (nVLP), and neither are prelabelled prior to screening. Duplicate arrays were run for the insect and plant VLP, and four negative control arrays were incubated with the buffer alone. After one hour incubation, slides were washed three times in TBST and three times in diH2O.

Polyclonal rabbit anti-nVLP was diluted 1:5000 in incubation buffer and 450 μl was added to the four VLP arrays and two of the negative controls. Incubation buffer only was added to the other two negative controls. All arrays were incubated for another hour and washed as above. ALEXA FLUOR® 555 labeled Goat Anti-Rabbit (INVITROGEN®) was diluted 1:2000 in incubation buffer and 450 μL was added to all eight arrays, which were then incubated for one hour and washed as above. Arrays were dried by centrifugation, and scanned on the PROSCAN ARRAY™ (PERKIN ELMER®) scanner at 555 nm and 70 PMT.

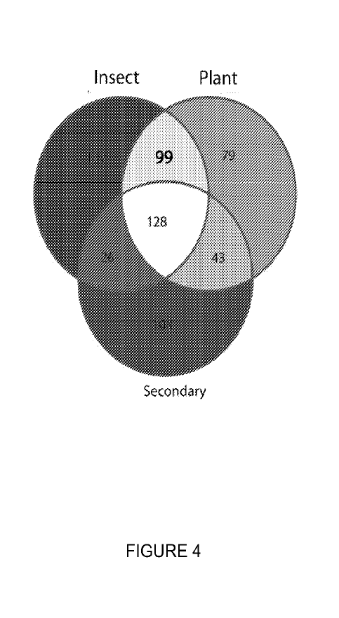

This screen identified 99 peptides that bound nVLP from both expression systems in the presence of insect and plant cell lysates (FIG. 4). We identified multiple peptides that bound nVLP when expressed in plants, peptides that bound nVLP in insect lysate, and peptides that bound independent of background matrix.

We filtered the list of peptide candidates by first sampling each candidate by MALDI MS to ensure that full-length peptide was present for that spot on the array; second by examining the binding intensity of each peptide for nVLP binding regardless of competitor; and finally, we also filtered the list by pI, hydrophobicity, solubility, and the predicted difficulty of synthesis of the sequence. We used these filters to select 6 peptides for further downstream studies of nVLP purification.

| Peptide | Sequence (SEQ ID NO:) |
|---|---|
| VP1 | LLYNKTFPHGRWSPSYPGSC |
| | (SEQ ID NO: 1) |
| VP2 | DWARSNTSRSMDFNLGWGSC |
| | (SEQ ID NO: 2) |
| VP3 | SFTFNWLKTDSKSGMHGGSC |
| | (SEQ ID NO: 3) |
| VP4 | LFFNIWPRRDPYWPAAWGSC |
| | (SEQ ID NO: 4) |
| VP5 | YIGTQIRVHWPANPHPVGSC |
| | (SEQ ID NO: 5) |
| VP6 | RWHRVDLRSHTELPRYIGSC |
| | (SEQ ID NO: 6) |

Coupling to Support: All 6 candidate peptides were synthesized by solid-phase peptide synthesis using standard FMOC chemistry and subsequently cleaved off the resin. The candidate peptides were attached to an iodoacetyl functionalized affinity matrix at a specifically determined level. The benefits of coupling our peptides to the affinity matrix as opposed to direct synthesis onto the bead are that it allows for greater control of the levels of peptide that are immobilized. The suitability of a ligand for affinity chromatography depends on its ability to bind the target molecule specifically and reversibly with an adequate affinity. We have shown that high levels of peptide immobilized on the affinity matrix, leads to a higher apparent affinity to the target molecule as a result of avidity. This leads to an increased difficulty eluting the target off the column and the subsequent use of harsher elution conditions (data not shown). For depletion protocols this feature is an advantage. Conversely, too low an amount of peptide on the affinity matrix leads to poor separation of the target antigen from the mixture and reduced column capacity. By varying the levels of peptide on the bead it is possible to find the immobilization level that allows sufficient affinity to the target to allow separation of the complex mixture but which also allows for the release of the target under mild elution conditions. In this examplification of the technology, mild conditions were important because nVLP's can dissociate under harsh conditions.

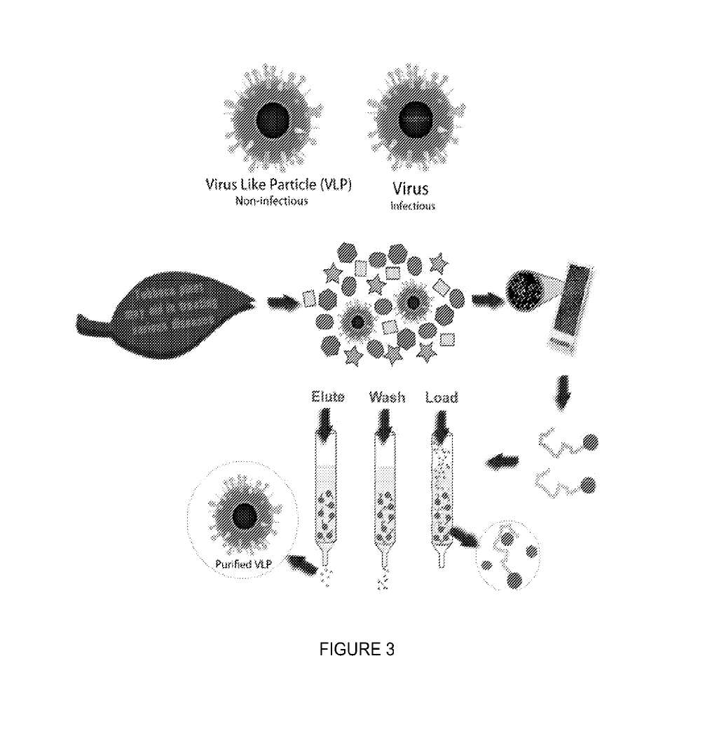

Norovirus Purification: Each peptide column was tested for its ability to capture and elute nVLP from tobacco cell lysate, however one peptide, referred to as nVLP1 was considered to be our optimal ligand (SEQ ID NO: 1). As can be observed in FIG. 3, an affinity column consisting of nVLP1 immobilized on a bead is able to capture nVLP from cell lysate. The captured nVLP was subsequently eluted using a mild elution buffer of 1M NaCl pH 7.4. From the examination of the Coomassie-stained gel, it appears that the eluted material was >90% pure, which was confirmed by silver staining (data not shown).

The collected fractions were combined, concentrated and probed via Western Blotting against a rabbit anti-nVLP which produced a band of the expected size, 58 kDa. The presence of a minor protein band at ˜54 kDa was noted in varying amounts; this corresponds to a proteolytic degradation product of VP1 and is also recognized by the anti-VP1 antibody (FIG. 4). The appearance of this product is the result of sample storage during this column development process, and can be avoided by rapid cell extract processing.

Examination of the eluted fractions obtained from the column by negative staining electron microscopy revealed the presence of intact nVLP's. Particles of ˜38 nm in diameter were clearly visible. 38 nm is the reported size of the nVLP produced in insect cells or plants (not shown), and this showed that the selected elution conditions did not cause the nVLP to dissociate, but that the nVLP remained intact.

To test the recovery yield of the column, cell lysate containing ˜500 μg of VP1 (as measured by SDS-PAGE) was loaded on the column and ˜470 μg of VP1 was recovered for a 94% yield. The dynamic binding capacity of the chromatography media was determined and is defined as the amount of target protein that the media binds under actual flow conditions before significant breakthrough of unbound protein occurs. As this parameter reflects the impact of mass transfer limitations that may occur as flow rate is increased, it is much more useful in predicting real process performance than a simple determination of saturated or static capacity.

Briefly, a large volume of VP1 containing lysate was prepared and its absorbance at 280 nm measured. This solution was continually loaded onto the column and the flow through measured for absorbance. The point at which breakthrough occurred, defined as 10% absorbance of the lysate, was used to calculate the binding capacity. The dynamic binding capacity of the column with a flow rate of ˜100 μL/min was 1.84 mg/mL. The static binding capacity of the column was determined by incubating the column with lysate overnight and measuring the amount of VP1 recovered. The static binding capacity for this material was 4 mg/mL of VP1.

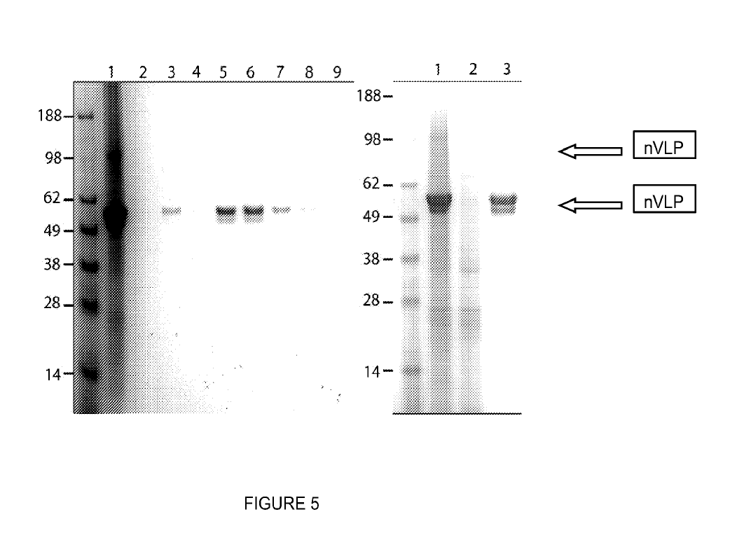

Noroviruses bind to histo-blood group antigens (HBGAs), which are carbohydrates that contain structurally related saccharide moieties, wherein type 1 and type 2 carbohydrate core structures constitute antigenically distinct variants. Several, but not all, noroviruses specifically bind to HBGAs, which are believed to function as receptors for docking and entry into the cell during infection. As previously mentioned, peptide nVLP1 was selected to specifically bind to G2.4 Minerva nVLPs. G2.4 Minerva is a global epidemic genotype and has been shown to bind to more HBGAs than any other genotype and as a result the majority of viral outbreaks are caused by this genotype. Experiments with recombinant VLPs have demonstrated that binding to HBGAs is highly strain-specific with at least 8 different binding patterns have been recognized. Interestingly our lead peptide nVLP1 has been shown to bind G1.1 nVLP produced in insect cells and could be eluted under the same conditions as G2.4 nVLP (FIG. 5). We therefore have identified at ligand that has cross reactivity to G1 and G2 capsid proteins in VLPs, with homology between genogroups I and II around 50%.

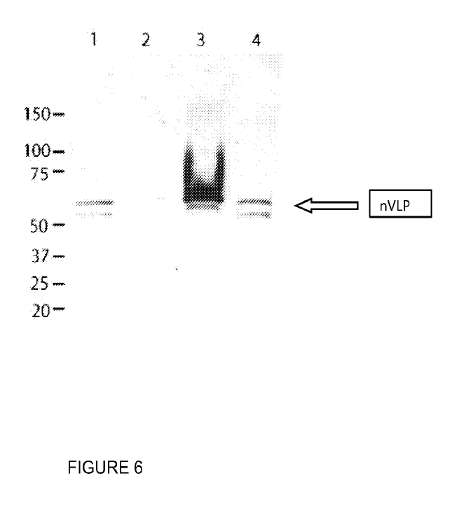

The invention developed affinity material that can purify intact nVLP in a single step (FIG. 6), with both high purity and yield and offers the potential to significantly reduce cost associated with the purification of norovirus or nVLPs.

The following references are incorporated by reference herein in their entirety:

Ball, J. M., et al., Cell. Biochem. Suppl. 19A: J1-200 (1995).

Bellofiore, P., et al., Identification and refinement of a peptide affinity ligand with unique specificity for a monoclonal anti-tenascin-C antibody by screening of a phage display library, J. Chromatog. A 1107(1-2): 182-191 (2006).

Giorgio Fassina, et al., Protein a mimetic peptide ligand for affinity purification of antibodies. J. Molec. Recognition, 9(5-6): 564-569 (1996).

Green, K. Y., et al, Clinical Microbiol. 31, 2185-2191 (1993).

Greying, M., et al., Creating high affinity from low affinity peptide ligands via thermodynamic additivity. Proc. Natl. Acad. Sci. USA (submitted) (2009).

Fassina, G., Oriented immobilization of peptide ligands on solid supports. J. Chromatog. A., 591(1-2): 99-106 (1992).

Jiang, X., et al., J. Virol. 66, 6527-6532 (1992).

Prasad, B.V.V., et al., J. Virol. 68, 5117-5125 (1994).

Williams, B., et al., Creating Protein Affinity Reagents by Combining Peptide Ligands on Synthetic DNA Scaffolds. J. Am. Chem. Soc. (accepted) (2009).

What is claimed is:

1. A method of selecting peptide ligands, said method comprising
a) obtaining a microarray having a library of candidate peptide ligands of length≧15 mers spatially addressed on said microarray;
b) screening said microarray with a target protein and optionally a competitor; and
c) detecting binding of said target protein and optionally said competitor to said microarray;
wherein high target protein binding, optionally divided by competitor binding, at a particular location on said microarray identifies a peptide ligand for said target protein.

2. The method of claim 1, wherein said candidate peptide ligands are of length≧17 mers.

3. The method of claim 1, wherein said candidate peptide ligands are of length≧20 mers.

4. The method of claim 1, wherein said library comprises 1000-10,000 different candidate peptide ligands.

5. The method of claim 1, wherein said library comprises 1000-10,000 different 20 mer candidate peptide ligands.

6. The method of claim 1, wherein the location and identity of each candidate peptide ligand on said microarray is predetermined.

7. The method of claim 1, wherein the location of each candidate peptide ligand on said microarray is predetermined, but the identity of each peptide ligand is unknown, and wherein said method further comprises d) sequencing the identified peptide ligand.

8. The method of claim 1, wherein said screening with said target protein and said competitor are done at the same time.

9. The method of claim 1, wherein said screening with said target protein and said competitor are done sequentially.

10. The method of claim 1, wherein said target protein and said competitor are combined at the time of said screening and repeating step b) with the target protein combined with a second competitor.

11. The method of claim 1, wherein said target protein is screened in a first cell lysate, and again in second different cell lysate, and wherein high target protein binding in both lysates at a particular location on said microarray identifies a peptide ligand for said target protein.

12. The method of claim 1, wherein the competitor is selected from the group consisting of lysates of a bacterial cell, a plant cell, a unicellular eukaryotic cell, a mammalian cell and an insect cell.

13. The method of claim 1, wherein detecting binding of said target protein to peptide ligands on the microarray is by antibody binding to said target protein.

14. The method of claim 1, wherein the target protein and competitor are labeled prior to said screening, and said labels are the same or different.

15. The method of claim 14, wherein said labels are one or more fluorescent dye or one or more isotopic radiolabel.

16. The method of claim 1, wherein the target protein is from norovirus.

17. The method of claim 16, wherein the target protein can self assemble into virus-like particles.

18. A peptide ligand for norovirus, said peptide ligand comprising SEQ ID NO: 1, 2, 3, 4, 5, or 6.

19. The peptide ligand for norovirus of claim 18, said peptide ligand comprising SEQ ID NO: 1.

20. The peptide ligand for norovirus of claim 18, wherein said peptide ligand has a modified backbone having increased stability over the unmodified peptide backbone.

21. A method of affinity purifying norovirus, comprising passing an impure mixture containing norovirus over a support matrix coupled to one or more peptide ligands of claim 18 under binding conditions, washing said support matrix, and eluting purified norovirus.

22. The method of claim 21, wherein said purified norovirus is at least 90% pure.

23. The method of claim 21, wherein said purified norovirus is at least 95% pure.

24. The method of claim 21, wherein said peptide ligand comprises SEQ ID No. 1.

25. A method of affinity purifying a target protein, comprising passing an impure mixture containing target protein under binding conditions over a support matrix bound to one or more peptide ligands, said peptide ligands identified by the method of claim 1; washing said support matrix; and eluting purified target protein.

26. A peptide ligand prepared by method of claim 1.

27. A norovirus detection method, wherein a sample is contacted with a peptide ligand of claim 17 under binding conditions, said sample is washed to leave only specific binding of said peptide ligand to said norovirus, wherein detecting specific binding of said peptide ligand to said sample indicates the presence of norovirus.

28. The norovirus detection method of claim 27, wherein said peptide ligand is coupled to a solid support.

29. The norovirus detection method of claim 27, wherein said peptide ligand is coupled to a solid support, and said norovirus binding is detected with an antibody.
